(19) United States
(12) Patent Application Publication
(10) Pub. No.: US 2008/0027554 A1
(43) Pub. Date: Jan. 31, 2008

(54) KIT AND METHODS OF TREATMENT OF AN INTERVERTEBRAL DISC

(76) Inventor: Karen D. Talmadge, Los Altos Hills, CA (US)

Correspondence Address: COOLEY GODWARD KRONISH LLP;ATTN: PATENT GROUP, Suite 1100, 777 - 6th Street, NW, WASHINGTON, DC, 20001, US

(21) Appl. No.: 11/830,294

(22) Filed: Jul. 30, 2007

Related U.S. Application Data
(60) Provisional application No. 60/834,284, filed on Jul. 31, 2006.

(51) Int. Cl. A61F 2/44

(52) U.S. Cl. 623/17.16; 604/506; 606/61

(57) ABSTRACT

Methods and kits for performing minimally invasive medical procedures are disclosed herein. In one embodiment, a method includes injecting an enzyme that is less immunogenic than chymopapain into an interior portion of an intervertebral disc. The enzyme can be mixed with a buffer prior to, during or after the injecting. The enzyme is configured to dissolve tissue within the interior portion of the intervertebral disc. Tissue that is dissolved by the enzyme can be removed from the interior portion of the intervertebral disc after the injecting. In some embodiments, the enzyme is a serine protease having an ability to sensitize that is less than chymopapain's ability to sensitize.

### CROSS-REFERENCE TO RELATED APPLICATIONS

This application claims priority to U.S. Provisional Patent Application Ser. No. 60/834,284, entitled “Kit and Methods of Treatment of an Intervertebral Disc,” filed Jul. 31, 2006, the disclosure of which is hereby incorporated by reference in its entirety.

### BACKGROUND

The invention relates generally to medical devices and procedures, and more particularly to a medical device for use in medical procedures associated with a spine.

Known medical devices are configured to percutaneously access an intervertebral disc to perform a variety of different medical procedures. Some known medical devices are configured to remove tissue from within the interior of an intervertebral disc. Other known medical devices are configured to provide some type of cutting means to tear or loosen tissue within the intervertebral disc. Thus, most medical devices and procedures for removing tissue from within an intervertebral disc involve the use of mechanical instruments to assist in cutting the tissue to prepare the tissue for removal.

Other known medical procedures involve the use of an enzyme to help break-down and remove tissue from within a body of a patient. For example, enzymes are known for breaking-down or degrading tumors, and for use in liver transplantation procedures. Another known enzyme-based medical procedure is the use of chymopapain in disc chemonucleolysis procedures. This procedure, however, has known possible negative side effects including possible death of the patient due to an allergic reaction.

Thus, a need exists for improved methods of using an enzyme to dissolve tissue within an intervertebral disc to assist in the removal of tissue from within an intervertebral disc.

### SUMMARY OF THE INVENTION

Methods and kits for performing minimally invasive medical procedures are disclosed herein. In one embodiment, a method includes injecting an enzyme that is less immunogenic than chymopapain into an interior portion of an intervertebral disc. The enzyme can be mixed with a buffer prior to, during or after the injecting. The enzyme is configured to dissolve tissue within the interior portion of the intervertebral disc. Tissue that is dissolved by the enzyme can be removed from the interior portion of the intervertebral disc after the injecting. In some embodiments, the enzyme is a serine protease having an ability to sensitize that is less than chymopapain's ability to sensitize.

### BRIEF DESCRIPTION OF THE DRAWINGS

FIG. 1 is a schematic representation of a kit according to an embodiment of the invention.

FIG. 2 is a side view of an intervertebral disc of a patient disposed between adjacent vertebrae and a delivery device according to an embodiment of the invention shown partially inserted within a portion of the disc.

FIG. 3 is a side view of the intervertebral disc of FIG. 2 shown with a portion of the nucleus pulposus removed.

FIG. 4 is a top view of an intervertebral disc shown in cross-section and an adjacent vertebra, illustrating an example propagation of serine protease (shown in radial dotted-lines) after being injected into the nucleus of the intervertebral disc.

FIG. 5 is a side view of an intervertebral disc disposed between adjacent vertebrae and a delivery device according to another embodiment of the invention shown partially inserted into a portion of the disc.

FIG. 6 is a top view of an intervertebral disc and a medical device according to another embodiment of the invention shown partially inserted into a portion of the disc.

FIG. 7 is a cross-sectional view of the medical device of FIG. 6 taken along line 7-7 in FIG. 6.

FIG. 8 is a flowchart illustrating a method according to an embodiment of the invention.

FIG. 9 is a flowchart illustrating a method according to another embodiment of the invention.

### DETAILED DESCRIPTION

Devices and methods for performing a medical procedure on a spine are described herein. In one embodiment, a method includes injecting a serine protease having an antigenicity less than an antigenicity of chymopapain into an interior portion of an intervertebral disc. The serine protease is configured to dissolve tissue within the interior portion of the intervertebral disc. Tissue that is dissolved by the serine protease can then be removed from the interior portion of the intervertebral disc after the injection. In some applications, a serine protease having an ability to sensitize less than an ability to sensitize of chymopapain is utilized in the injection. In some embodiments, the serine protease includes subtilisin.

In another embodiment, a kit includes a medicament including a serine protease having an ability to sensitize that is less than an ability to sensitize of chymopapain. The serine protease is configured to dissolve tissue within a nucleus of an intervertebral disc. The kit includes a delivery device, such as a syringe, configured to be percutaneously inserted into the nucleus of the intervertebral disc and inject the medicament into the nucleus of the intervertebral disc. In one variation, the delivery device is configured with a pressure and/or volume monitor to allow the physician to verify the pressure being applied during the injection and the volume of medicament injected into the intervertebral disc. The medicament may be preloaded into the delivery device or it may be provided to the physician in a separate container along with the delivery device. The kit may further include instructions for insertion of the delivery device into the intervertebral disc. The instructions may also include dosing information for the delivery of the medicament. The instructions may further include timing information on the delivery of the medicament and removal of the dissolved tissue. In some embodiments, the serine protease has an antigenicity that is less than an antigenicity of chymopapain. In some embodiments, the serine protease is subtilisin.

In another embodiment, a structure includes an annulus of an intervertebral disc and a nucleus of an intervertebral disc at least partially bounded by the annulus of the intervertebral disc. The nucleus includes nucleus pulposus dissolved through surgical intervention with a delivery of an enzyme, such as a serine protease having an antigenicity less than an antigenicity of chymopapain into the nucleus of the intervertebral disc. The nucleus can include a first portion with the nucleus pulposus dissolved through surgical intervention and a second portion with undissolved nucleus pulposus. The structure can define a volume having the dissolved nucleus pulposus and bounded by the annulus. In some embodiments, the structure can further include a portion of the enzyme (e.g., serine protease) remaining after the nucleus pulposus has been dissolved and disposed within the volume. In some embodiments, the nucleus includes a first portion with the nucleus pulposus dissolved through surgical intervention and a second portion with undissolved nucleus pulposus and the structure defines a volume having the first portion of the nucleus pulposus and bounded by at least one of the second portion of the nucleus pulposus or the annulus. In some embodiments, the enzyme is subtilisin.

A structure as described herein can be formed in vivo, with the intervertebral disc disposed within a spinal column of a patient, or in vitro. It may be desirable to inject an enzyme within an interior of an intervertebral disc that is not disposed within a body. For example, an intervertebral disc, received from an organ donor (e.g., cadaver, animal), can be injected with an enzyme to dissolve a portion of the nucleus pulposus tissue as described herein. Such a modification to an intervertebral disc in vitro, may be desired to prepare the disc for implantation into a recipient patient.

In some embodiments, a serine protease having an antigenicity less than an antigenicity of chymopapain can be used for the manufacture of a medicament for treatment of an intervertebral disc. The medicament can be administered by delivering the medicament into a nucleus of an intervertebral disc. The delivery can be minimally invasive and the medicament can be configured to dissolve at least a portion of pulposus within the nucleus of the intervertebral disc. The dissolved pulposus and a remaining portion of the medicament can collectively be removed from the nucleus of the intervertebral disc after the injecting.

As used in this specification and the appended claims, the singular forms “a,” “an” and “the” include plural referents unless the context clearly dictates otherwise. Thus, for example, the term “a lumen” is intended to mean a single lumen or a combination of lumens. A medicament can include a single medicament or a mixture of a combination of medicaments. Furthermore, the words “proximal” and “distal” refer to direction closer to and away from, respectively, an operator (e.g., surgeon, physician, nurse, technician, etc.) who would insert the medical device into the patient, with the tip-end (i.e., distal end) of the device inserted inside a patient's body. Thus, for example, an end of a medical device inserted inside the patient's body would be the distal end of the medical device, while the end of the medical device outside the patient's body would be the proximal end of the medical device.

Serine proteases are known protein-digesting enzymes and include, for example, subtilisin and chymopapain. Each of these serine proteases have an associated antigenicity. Some of the serine proteases have an antigenicity less than the antigenicity of chymopapain. Subtilisin is one such serine protease. Although the description herein at times refers to only subtilisin, it should be understood that other enzymes, such as serine proteases, having an antigenicity less than the antigenicity of chymopapain are also applicable. Thus, various enzymes that are less immunogenic than the enzyme chymopapain can alternatively be used.

Subtilisin, or carbonyl hydrolases, are alkaline proteases. Subtilisin is secreted by members of the genus Bacillus and can be produced by bacteria. Subtilisin is also included within the serine protease group of enzymes and is inhibited by diisopropyl fluorophosphate. Serine proteases have properties that make them desirable, for example, to the detergent industry. Serine proteases can exhibit high specific activity on proteinaceous substrates, can function optimally at moderate temperatures, and are stable under alkaline conditions. Subtilisin gains part of its stability by binding to calcium. Engineered versions of subtilisin have retained the stability and protease activity by replacing the calcium site with a disulfide bond. Subtilisin catalyzes the breakdown of proteins into polypeptides and resembles trypsin in its action.

A known method of treating a herniated intervertebral disc includes the injection of the enzyme chymopapain into the nucleus of the disc. This procedure is referred to as chymopapain chemonucleolysis. Chymopapain is a serine protease and is more specifically categorized as a cysteine proteinase similar to papain in specificity. The chymopapain dissolves the nucleus pulposus tissue within the intervertebral disc while having substantially no effect on the surrounding annulus fibrosis. Unfortunately, there have been cases of severe allergic anaphylactic reaction, including death, as a result of the chymopapain escaping from the intervertebral disc into other areas within a patient's body. This can occur, for example, when the annulus fibrosis is damaged or has ruptured prior to the treatment. Thus, during the treatment, chymopapain can leak through the ruptured or damaged region. Leakage of chymopapain can also occur over time when the enzyme is not removed after placement in the nucleus of a disc. Such allergic reactions can also occur when the enzyme chymopapain is improperly placed, such as when chymopapain is placed within the subarachnoid space (e.g., part of the nerve root and spinal cord).

The chymopapain is typically delivered in a liquid solution making it even more susceptible to leaking, and is left in the disc to act over time. The chymopapain solution is also typically not radiopaque making the path and/or location of the enzyme difficult to identify when the solution has leaked from the disc. Another problem with chymopapain is that the enzyme is sensitizing, which means that it primes the immune system in susceptible patients so that a second exposure can lead to an allergic reaction (e.g., anaphylaxis). Because of these issues, the use of chymopapain in the spine has been largely avoided.

Here, minimally-invasive medical procedures are provided that use other enzymes, such as other enzymes from the serine protease family (including new formulations of chymopapain) to break-down the nucleus pulposus within an intervertebral disc that are formulated for improved results. For example, serine proteases can be formulated for increased viscosity so that they do not easily leak out of the disc. Such formulations can include, for example, glycerol, polyethylene glycol, sucrose, mannitol or other fillers that increase viscosity. In addition, the enzymes and or its carrier solution can be formulated with a contrast medium, to allow visibility under X-ray imaging. The enzymes can be delivered at a dosage that will allow complete digestion within a specified period of time, such as, for example, 30 minutes (the dosage can vary for different enzymes, different formulations, and may also be dependent on the physical condition of the disc). For example, the dissolution time can be between 5 and 180 minutes depending on the particular situation. It is desirable to provide sufficient enzyme to adequately disrupt at least a portion of the nucleus so that it can be removed, for example, with suction alone, with suction after further disruption using a mechanical tool (such as a rongeur), or with other nucleus removing methods.

Another minimally-invasive medical procedure is provided that uses a serine protease having a similar substrate specificity as chymopapain, but a reduced potential to cause sensitization, such as the enzyme subtilisin. Subtilisin can be used to break-down or dissolve tissue within an interior portion of an intervertebral disc and does not present the allergy-related issues associated with the use of chymopapain. Thus, even if the subtilisin were to leak out of the disc, the patient would not be at risk of the same allergic reactions that are associated with chymopapain. For example, subtilisin has a similar substrate specificity as chymopapain, but with reduced antigenicity and a reduced ability to sensitize compared to chymopapain. Subtilisin's primary sequence and tertiary structure are different from chymopapain, which greatly reduces its potential for sensitization. In some embodiments, the enzyme can be substantially non-sensitizing. Other serine proteases that can be used include digestive enzymes (e.g., papain, trypsin, elastase, etc.), the clotting factors (Factor X, Factor XI, thrombin and plasmin), and several proteases from the complement cascade (C1r, C1s, C4b,2a and C3b,Bb).

Methods and devices are described herein that can be used in conjunction with the injection of an enzyme. For example, a device can be used that detects whether the nucleus is in fluid communication with the subarachnoid space. Such fluid communication between the nucleus and the subarachnoid space can be caused, for example, by improper needle placement of an injection needle. In some embodiments, methods and devices are provided to remove the enzyme after a specified time period to reduce the likelihood of the enzyme leaking out of the intervertebral disc. In some embodiments, methods and devices are provided to assess when a procedure is completed. In some embodiments, an optional contrast media, such as a radiopaque liquid, can be provided for delivery into the intervertebral disc through, for example, a delivery needle prior to injecting the enzyme treatment. The contrast media can be used to confirm proper needle placement in the disc, and confirm the lack of contiguity between the nucleus and the subarachnoid space (e.g., the lack of fluid communication between the nucleus and the subarachnoid space) prior to enzyme delivery.

In some embodiments, a kit is provided that includes various devices to inject an enzyme into an intervertebral disc. For example, a kit can include an outer needle configured to puncture the skin of the patient and reach the area proximate the outside of the disc, but not enter into the annulus or interior of the disc. In such an embodiment, an optional inner delivery needle can also be included. The inner delivery needle can be used to puncture the annulus and reach the nucleus pulposus. Such an inner delivery needle can also be used to inject an enzyme into the disc. Using the same delivery needle for entering the disc and injecting the enzyme can increase sterility and reduce the risk of disc infection. A guidewire can also optionally be included. The guidewire can be used to help facilitate needle placement. In some alternative embodiments, a single delivery needle can used rather than an inner delivery needle and an outer delivery needle.

A kit can also include one or more enzymes. Such enzymes can be lyophilized. The methods for selecting one or more enzymes from the kit during a medical procedure is described below. The kit can also include one or more buffers to be mixed with the enzyme to define a solution appropriate for digestion of the nucleus. The kit can also include optional thickening agents, such as sucrose or polyethylene glycol, to thicken the enzyme solution, and optional agents for making the enzyme solution radiopaque, such as iodine salts or gadolinium. In some embodiments, the kit can include an optional second buffer configured to stop the enzyme action when the medical procedure is completed. Such an optional second buffer can function, for example, through pH or ionic charges, or the addition of chelating agents, that stop enzyme action.

In some embodiments, a normal or threaded syringe can be used to deliver a buffer into the enzyme (within a separate container) and then the dissolved enzyme solution can be loaded into the delivery needle. In one embodiment, the buffer is a gel-like material, and the enzyme is mixed with the gel prior to injection into the patient's body. The mixing can occur, for example, by having an enzyme disposed in a syringe and suctioning the gel material into the syringe to mix the gel with the enzyme. In another embodiment, the syringe can include a luer-lock connector for engaging a catheter or cannula/needle that has been positioned with the distal end of the cannula inside the disc. In some embodiments, the enzyme and the buffer can be injected into a container for mixing. In another embodiment, the enzyme and buffer can each be injected through a separate end of a Y adaptor, such that the enzyme and the buffer mix as they enter into the catheter during transport into the intervertebral disc. In yet another embodiment, the buffer and enzyme can be premixed at a manufacturing site and provided as a single mixture. In this approach, other components, such as an optional preservative can be included in the enzyme-buffer mixture. Such a mixture can be stored in a refrigerated environment. The particular buffers used can depend on the particular enzyme chosen for the medical procedure.

The selection of a particular enzyme can be dictated by the enzyme's ability to digest the nucleus pulposus without substantially affecting the annulus of the disc. In some embodiments, the optimal enzyme is less immunogenic than chymopapain and will be able to be delivered into the disc space in a concentration sufficient to complete digestion within a reasonable time period. For example, in some cases the time period for digestion will be no more than 30 minutes, in other embodiments it can be, for example, between 5 and 180 minutes. Such enzymes can include, for example, subtilisin, collagenase and chondroitinase ABC. Methods for determining whether an enzyme has a low immunogenicity can include, for example, finding isolates that have never been used in humans, and/or modifying the amino acid sequence of the enzymes to reduce immunogenicity using for example, epitope analysis or genetic engineering techniques.

In some embodiments, a solution is provided for dissolving the enzyme where the solution promotes enzyme action through appropriate ingredients (e.g., salts, buffers to provide the correct pH, etc.). Such solutions can also be (1) optionally viscous, and therefore, less likely to flow out of the nucleus, and (2) optionally radiopaque, and therefore, visible under X-ray imaging during delivery into the nucleus. In one variation, the pH of the carrier solution can be changed from neutral to basic, so that if it does leak out of the disc, the solution will not be able to digest other tissue within the patient's body.

A gel-type buffer, although not necessary for carrying the enzyme, can be a useful carrier to minimize leakage of the enzyme from the intervertebral disc and into the space outside the intervertebral disc. Other types of carrier solutions can alternatively be used, such as, albumin, ethylene glycol, and polyvinyl glycol. Also, as many enzymes are active around 37 degrees Celsius, it may be desirable to keep the gel buffer material at around 37 degree Celsius for later mixing with the enzyme.

The choice of a buffer or buffers can be dependent on the particular enzyme to be used for the medical procedure. In one example, an enzyme that is reactive around pH 7 can be used. In such an example, a first buffer with a buffering region around pH 7 may be used to carry the enzyme and keep the enzyme active. The buffer can preserve the enzyme and prevent the enzyme from denaturing. In this example, a second buffer can have a buffering region that is more basic (e.g., pH 8). For example, a buffer containing ammonium salt can be introduced into the nucleus of the intervertebral disc to stop enzymatic action by moving the inner-disc environment out of the desired enzyme reactive range. Many enzymes have an optimal functional range that is neutral (pH 7) or slightly acidic. Alternatively, a chelating agent can be applied to stop the enzyme reaction. For example, the chemical compound ethylenediaminetetraacetic acid (EDTA) can be introduced into the intervertebral disc to stop the enzymatic reactions.

In another variation, a buffer can be provided in the form of a time-release capsule. For example, a buffer having a pH 8 can be incorporated into a time-release capsule and mixed with the enzyme. The enzyme solution (with the buffer) can then be injected into the interior of an intervertebral disc. The time-release buffer can be configured to be released within the interior of the intervertebral disc (and mixed with the enzyme) after a specified time period to change the reaction of the enzyme. For example in some embodiments, a time-release buffer can be configured to stop the enzymatic action of the enzyme. In some embodiments, an enzyme can be injected that is inactive, and the time-release buffer can be configured to cause the enzyme to become active.

Other methods or devices to disrupt partially digested nucleus tissue include, for example, the use of ultrasound. This type of procedure can be performed by direct insertion of an ultrasonic transducer into the disc. Such a procedure can also be performed by injecting gas microbubbles into the disc (either at the same time as the injection of the enzyme or afterwards) and then applying ultrasound energy through the skin. The ultrasound energy causes the microbubbles to undergo cavitation, which in effect creates micro-implosions that helps to disrupt the nucleus tissue.

As mentioned previously, in some embodiments, agents can be added to the enzyme solution or gel to allow them to be visualized intra-operatively while being delivered. In such a case, the flow and distribution of the enzyme solution or gel can be monitored with an imaging device, such as under X-ray fluoroscopy. Examples of such agents can include barium, iodine, and other radiopaque salts, in powder or liquid form.

A device to provide suction can also be provided to remove the enzyme solution when the dissolution process is complete. For example, a bulb to create suction can be coupled to the delivery needle, or a delivery needle can be coupled to a suction device with, for example, tubing. Other devices, such as a rongeur, can also be provided to help disrupt partially digested nucleus pulposus, if desired in some embodiments. In addition, an optional endoscopic camera can be inserted through the outer delivery needle to assess whether the digestion is complete. Alternatively, an optional endoscopic camera can be inserted through an optional second outer needle/cannula to provide continuous visualization of the space within the nucleus of the intervertebral disc.

Any of the combination or sub-combination of the various devices described herein can be included in a kit. The various devices can be used in various combinations and sub-combinations in a medical procedure within a spine. In one example method, an introducer cannula is inserted into the patient with the distal tip docked at the annulus of the intervertebral disc. A catheter is inserted through a lumen of the introducer cannula and penetrates through the annulus with the distal tip of the catheter positioned within the nucleus of the disc. An enzyme is then injected through the catheter and into the nucleus of the intervertebral disc. After the injection of the enzyme, the catheter can be withdrawn. Optionally, a scope or camera, such as a fiber, an endoscope, or a CCD camera, can be inserted into the lumen of the introducer cannula to monitor the enzymatic action inside the disc. In one variation, a drug pump can be attached to the catheter to deliver the enzyme solution into the intervertebral disc over a specified period of time. In another embodiment, the catheter is anchored in the intervertebral disc (e.g., via an expandable member like a balloon that is expanded at the distal tip of the catheter and within the annulus or nucleus of the intervertebral disc), the introducer catheter is removed, and a drug pump is then attached to the catheter to deliver the enzyme solution into the disc. Optionally, a drug pump can be programmed to deliver a deactivating agent into the disc to stop the enzymatic activity after a predefined period of time has elapsed.

In another example method, a first introducer cannula can be disposed adjacent the outside of the annulus of the intervertebral disc and configured to deliver an enzyme into the disc via a catheter or delivery needle. A second introducer cannula can be disposed at a second location adjacent the outside of the annulus; an endoscope can be introduced through this second introducer cannula to monitor the delivery needle placement through the first introducer cannula and the injection of enzyme through the needle. For example, a tip of the delivery needle can be moved as the enzyme is injected into the nucleus and the endoscope can provide feedback on the position of the delivery needle. In addition, the endoscope can be used to monitor continuously the condition of the nucleus as the injected enzyme dissolves the nucleus.

In another embodiment, an introducer cannula can be large enough to support simultaneous introduction of the enzyme delivery catheter/needle and the endoscope/optical fiber. For example, the introducer cannula can include a single lumen sufficiently large for such an application. Alternatively, the introducer cannula can include multiple lumens to accommodate various medical devices at the same time.

In some embodiments, a pressure inside the intervertebral disc can be monitored to provide information on the condition of the disc as the enzyme dissolves the nucleus. For example, a pressure monitor can be coupled to the catheter used to inject the enzyme to monitor the pressure inside the disc. In another embodiment, the catheter can include two lumens, one for delivery of the enzyme and the other for pressure measurement. In yet another embodiment, a micro-pressure sensor can be placed at the distal tip portion of the catheter or needle for monitoring the pressure inside the disc.

In some embodiments, an optical emitter and detector can be used to monitor and provide feedback on the condition of the intervertebral disc as the enzyme digests the nucleus of the intervertebral disc. For example, optical fibers can be introduced into the nucleus of the intervertebral disc to deliver excitation light and to capture the reflected or transmitted light back to a sensor. Alternatively, an infrared (IR) emitter and IR detector are inserted into the disc to make such a measurement. Spectral analysis can also be applied on the detected light signal to determine the condition of the nucleus.

As stated above, subtilisin, and other enzymes having an antigenicity less than an antigenicity of chymopapain, can be injected into an interior of an intervertebral disc where the subtilisin can dissolve or break-down nucleus pulposus tissue in the intervertebral disc. Although some embodiments are described with reference to the injection of the enzyme subtilisin, one of ordinary skill in the art having the benefit of this disclosure would appreciate that other enzymes can also be used as described herein.

The injection of subtilisin can be used to treat various disc disorders, such as a protruded disc or an extruded disc. In some embodiments, the dissolved nucleus pulposus tissue, and remaining subtilisin, can be left in the disc indefinitely. In other embodiments, the dissolved nucleus pulposus tissue, and remaining subtilisin, are removed from the intervertebral disc. Various known removal methods, such as using a catheter or cannula to access the interior portion of the intervertebral disc and a suction device to suction out the dissolved tissue and remaining subtilisin, can be used to remove at least a portion of the dissolved and/or broken-down tissues. Therefore, nucleus pulposus tissue can be removed from the intervertebral disc without using a mechanical instrument to cut and/or tear the tissue.

After the nucleus pulposus tissue is removed from the intervertebral disc, other optional medical procedures can be performed on the disc to replace the nucleus (e.g., implanting a prosthesis within the nucleus of the disc, injecting a biocompatible polymer into the nucleus region, etc.). Although the following description focuses on use of a serine protease, such as subtilisin in an intervertebral disc procedure, one of ordinary skill in the art having the benefit of this disclosure would appreciate that the methods and procedures described herein can be performed on other areas of a body, including other bone structures, collagen tissues, and soft tissue areas.

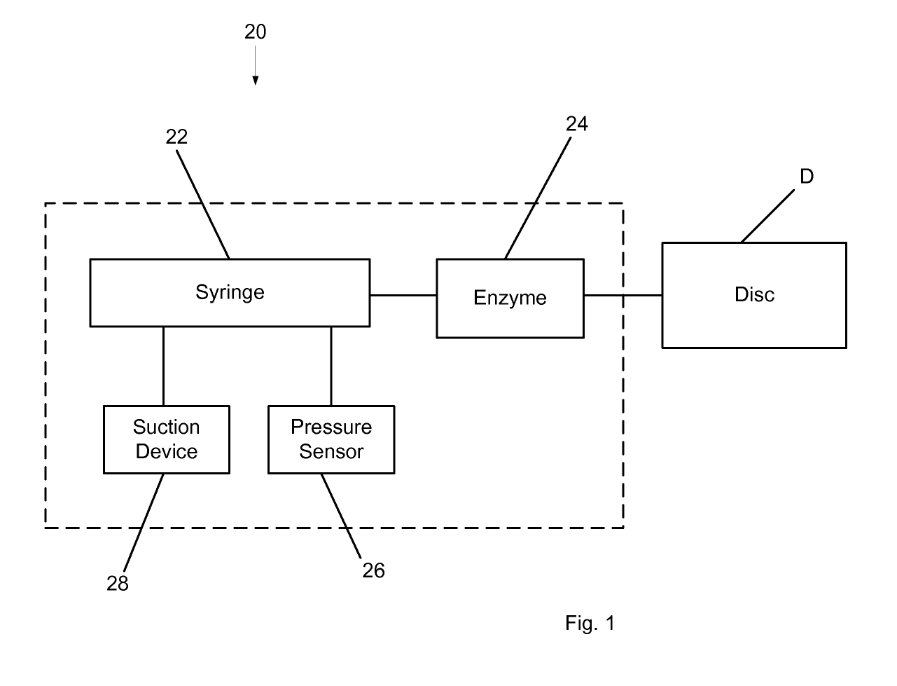

FIG. 1 is a schematic illustration of a kit according to an embodiment of the invention. A kit 20 can include an enzyme, such as a serine protease 24 having an ability to sensitize less than an ability to sensitize of chymopapain, and/or having an antigenicity less than an antigenicity of chymopapain, as described above (such as subtilisin), or a medicament including such a serine protease (also referred to herein collectively as serine protease 24 for convenience). The serine protease 24 can be used to dissolve nucleus pulposus tissue within a nucleus of an intervertebral disc D. The kit 20 can also include one or more delivery devices 22 (e.g., a syringe, catheter) for injecting the serine protease 24 into the nucleus of the intervertebral disc D. The delivery device 22 can include a needle (not shown in FIG. 1) and can have a variety of different configurations as appropriate. The amount of serine protease 24, or related medicament, to be injected can be determined based on the desired amount of tissue to be removed from the nucleus. For example, a predetermined amount of serine protease 24 can be configured to dissolve a predetermined amount of tissue within the intervertebral disc D. This allows the physician to select the amount of serine protease 24 to achieve the desired tissue removal.

The kit 20 can optionally include a pressure sensor 26 configured to measure the injection pressure and/or a pressure within the nucleus of the intervertebral disc D. The kit 20 can also optionally include a suction device 28 configured to extract dissolved tissue and remaining serine protease 24 from within the intervertebral disc D to a location outside of the patient's body, such as into a containment device (not shown).

In use, the serine protease 24 can be injected into the nucleus of the intervertebral disc D to dissolve at least a portion of nucleus pulposus tissue within the nucleus. The serine protease 24 can be formulated such that the serine protease 24 only dissolves the nucleus pulposus tissue and not the annulus fibrosis tissue of the intervertebral disc D. The dissolved nucleus pulposus tissue and remaining serine protease 24 can remain in the intervertebral disc D, or can alternatively be removed using, for example, the suction device 28. If the dissolved nucleus pulposus tissue is removed, a cavity (not shown in FIG. 1) can be formed within the nucleus of the intervertebral disc D, and a disc prosthesis can optionally be disposed within the cavity.

As stated above, the pressure sensor 26 can be used to measure a pressure within the nucleus of the intervertebral disc D. For example, a first pressure can be measured in the nucleus of the intervertebral disc D before injecting the serine protease 24, and then a second pressure can be measured after the serine protease 24 has been injected. A difference between the first pressure and the second pressure may indicate the amount of nucleus pulposus tissue that has dissolved. In another example, when the nucleus pulposus tissue has sufficiently dissolved, the pressure within the nucleus may reach a threshold pressure value. In some embodiments, the determination as to whether the tissue has sufficiently dissolved is based on a specified lapse of time.

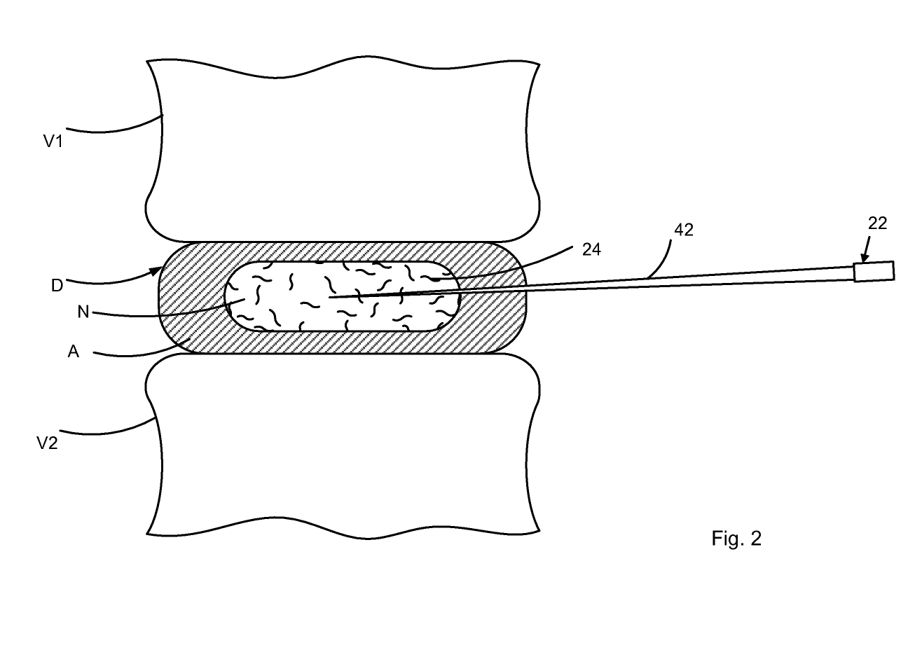

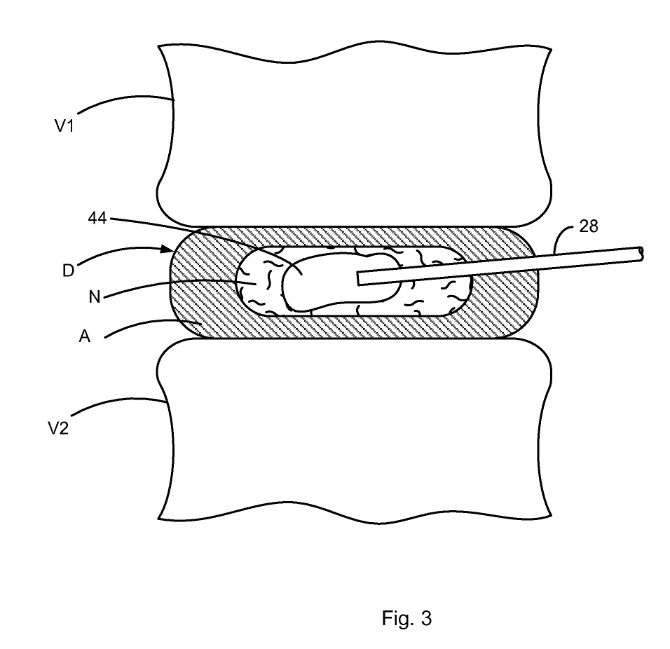

FIG. 2 is a side view of an intervertebral disc D disposed between a first vertebra V1 and a second vertebra V2. The disc D includes an annulus fibrosis A and a nucleus N including nucleus pulposus tissue. As shown, a delivery device 22, such as a syringe, includes a needle 42 and can inject serine protease 24 (as described above) or related medicament into the nucleus N of the intervertebral disc D. The serine protease 24 can dissolve at least a portion of the nucleus pulposus tissue. After the nucleus pulposus tissue has sufficiently dissolved, the dissolved nucleus pulposus tissue, and remaining serine protease 24, can be removed from the intervertebral disc D using, for example, a suction device 28 that can suction the dissolved tissue from within the intervertebral disc D, as shown in FIG. 3. In this example, the suction device 28 that can be coupled to a device (not shown) that can provide suction force to a lumen (not shown) of the suction device 28. The annulus A and/or remaining undissolved nucleus pulposus tissue forms a cavity or void 44 within the nucleus N, as shown in FIG. 3. In one variation, the distal end of a cannula is first inserted into the interior portion of the intervertebral disc and a suction device is connected to a proximal end of the cannula. The suction device can be implemented to generate a suction within a lumen of the cannula to remove the dissolved tissue from the intervertebral disc. In another variation, the cannula includes two or more lumens. In such an example, saline can be delivered into the intervertebral disc through a first lumen of the cannula while suction can be provided in a second lumen of the cannula to remove the dissolved tissue. To determine if the nucleus pulposus tissue has sufficiently dissolved, a pressure of the nucleus N may be measured and used as an indicator as described previously.

After the dissolved tissue (and remaining serine protease 24) has been removed from the nucleus N of the intervertebral disc D, other optional medical procedures can be performed. For example, a prosthesis (not shown) can be disposed within the cavity 44 formed by the removal of nucleus pulposus tissue in the nucleus N.

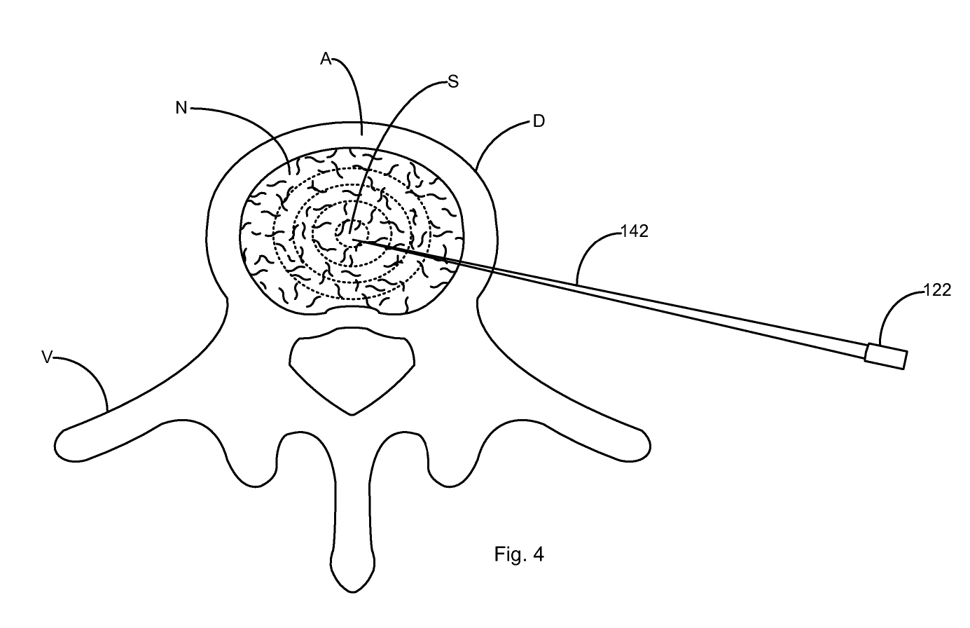

FIG. 4 is a top view of an intervertebral disc D (shown in cross-section) and an adjacent vertebra V. FIG. 4 illustrates an example propagation of the subtilisin (shown in radial dotted-lines) after being injected into the nucleus N of the intervertebral disc D. As shown, the subtilisin can be injected using a syringe 122 having a small gauge needle 142 percutaneously inserted from a posterolateral approach into the center of the disc D. This positioning allows the subtilisin to propagate substantially isotropicly from the injection site S. By allowing the subtilisin to remain within the disc D for a specified period of time, the size and location of the nucleus N dissolved by the subtilisin centered about the injection site S, can be controlled.

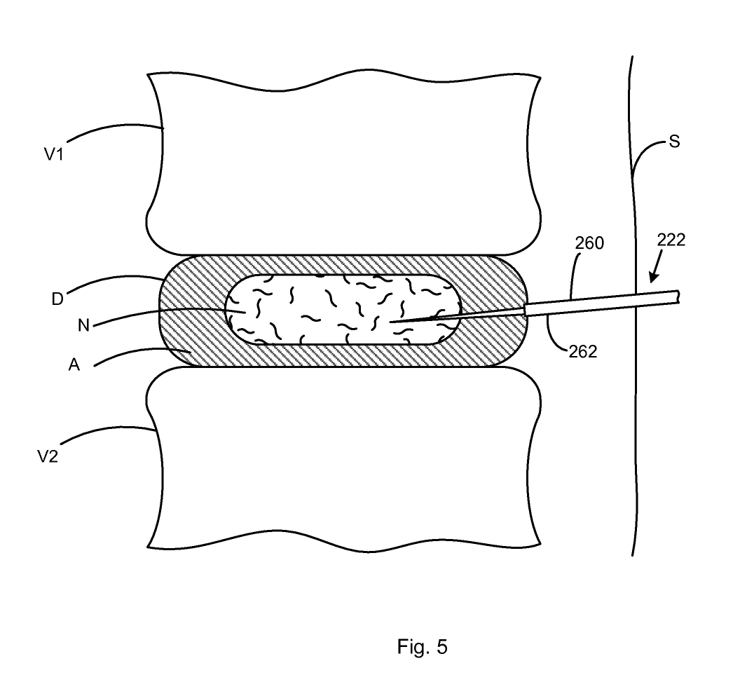

FIG. 5 is a side view of an intervertebral disc D (shown in cross-section) disposed between a first vertebra V1 and a second vertebra V2. In this embodiment, a medical device 222 used in the delivery of an enzyme to an interior of an intervertebral disc includes a first needle 260, a stylet (not shown) removably disposable within a lumen of the first needle 260, and a second needle 262 also removably disposable within the lumen of the first needle 260. The first needle 260 can be, for example, a blunt spinal needle (or other type of needle) that is long enough to reach the spine. The first needle 260 can also be referred to as a “guide” needle.

In this example, the procedure includes making a small incision through the patient's skin. The first needle 260 (with the stylet disposed within the lumen of the first needle 260) is then inserted through the incision in the skin and advanced, while under imaging control, to the surface of the disc D, but without penetrating the annulus A of the disc D. The gauge of the first needle 260 can be, for example, between 6 and 14, although larger and smaller gauge needles can alternatively be used. The inner stylet is removed from the lumen of the first needle 260, leaving behind the first needle 260, which now forms a passageway though the skin S to the outside of the disc D. A second needle 262 (e.g., a spinal needle) of a smaller gauge, is then brought though the lumen of the first needle 260 and into the disc D, also under image guidance. The second needle 262 can include a sharp tip, and can be configured to be moved or slide down the lumen of the first needle 260 (e.g., “guide” needle), emerging from the distal tip of the first needle 260 and into the disc D. The second needle 262 can be referred to as the “delivery” needle because it will be used to deliver the enzyme. For example, a syringe (not shown) filled with enzyme can be attached to a proximal end of the second needle 262 before or after the second needle 262 is positioned at least partially within the nucleus N of the disc D.

This “double needle” configuration can provide enhanced sterility because the needle that penetrates the disc D (e.g., second needle 262) does not touch the skin of the patient. The disc is an aseptic environment and even skin that has been prepared intra-operatively with sterilizing solutions can be a potential source of infection for the disc. Consequently, even body sites with a low bioburden, such as sterilized skin, can create a higher risk of infection in the disc than might be the case in most other sites in a body. Thus, the “double needle” configuration discussed in reference to FIG. 5 can provide enhanced sterility because the inner needle does not touch the skin of the patient.

The gauge of the “delivery” needle or second needle 262 can be as large as the size that just fits down the lumen of the “guide” needle or first needle 260 without friction, or can be much smaller. The second needle 262 can have a size, for example, of 8 gauge through 20 gauge. Thus, various gauge needles for the “delivery” needle (e.g., second needle 260) can be used to deliver the enzyme solution. Most solutions of protein can be delivered in smaller gauge needles (as well as the larger gauge needles), and gels are more likely to be delivered in the larger needle sizes described herein.

After the desired time period of dissolution or digestion of the enzyme has passed, the dissolved tissue and remaining enzyme solution within the intervertebral disc D can be removed as described previously. For example, the second needle 262 can be removed from the lumen of the first needle 260 and a separate device configured to provide suction force can then be inserted through the second needle 262. Alternatively, in some embodiments, the second needle 262 can be configured to remove the dissolved tissue and remaining enzyme solution from the disc D. For example, a device configured to provide suction force can be coupled to a proximal end portion of the second needle 262. The dissolved tissue and remaining enzyme solution can then be suctioned through the lumen of the second needle 262. In some embodiments, to provide suction force, a flexible bulb member (not shown) is coupled to the proximal end portion of the second needle 262. The bulb member can be manually actuated to create a suction force through the lumen of the second needle 262. The dissolved tissue and remaining enzyme solution can then be suctioned into an interior of the bulb member.

In some embodiments, as stated previously one or more buffers can be added to the enzyme prior to, after, or simultaneously with the enzyme being injected into a disc. In the example embodiment described above with reference to FIG. 5, a first buffer can be added to the enzyme to carry the enzyme and keep the enzyme active. For example, the first buffer can have a buffering region around pH 7 (e.g., neutral). After a desired time period of dissolution, a second buffer can then be injected into the intervertebral disc using, for example, a delivery needle similar to second needle 260. The second buffer can have, for example a buffering region that is more basic, e.g., around pH 8 (e.g., a buffer containing ammonium salt), which can stop the enzymatic action by moving the interior of the intervertebral disc out of the desired enzyme reactive range. Thus, the use of a buffer as described here can allow for the enzymatic action to be turned off after a desired time period. In some embodiments, an enzyme can be injected into an intervertebral disc that is inactive, and a buffer can be added later to cause the enzyme to become active.

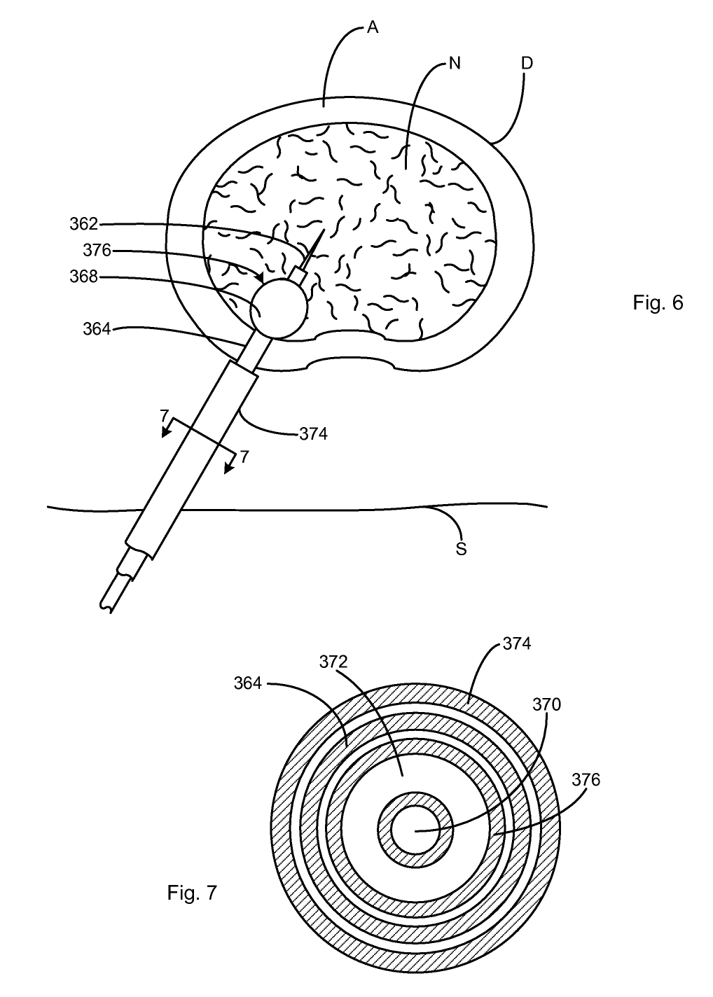

FIG. 6 illustrates another embodiment of medical devices used in the delivery of an enzyme solution to an interior of an intervertebral disc. In this embodiment, an introducer device 374 is inserted through an incision in a patient's skin S. A distal end of the introducer 374 is positioned adjacent an intervertebral disc D, but does not penetrate the annuls A of the intervertebral disc D. A catheter 364 having a stylet (not shown) extending through a lumen of the catheter 364, is then inserted through a lumen of the introducer 374. The stylet enables the catheter 364 to be able to pass through the annulus fibrosis A and into the nucleus N of the intervertebral disc D. The stylet can then be removed from the catheter 364, leaving the catheter 364 positioned to provide access to an interior of the intervertebral disc D.

With a distal end portion of the catheter 364 positioned within the intervertebral disc D, and a proximal end portion of the catheter 364 extending outside of the body, other devices can be passed through the lumen of the catheter 364 and into the intervertebral disc D. An anchoring cannula 376, having an expandable anchoring member 368 disposed at a distal end portion thereof, is passed through the lumen of the catheter 364 with the anchoring member 368 in a collapsed configuration (not shown). The anchoring member 368 is positioned within the intervertebral disc D and moved to an expanded configuration as shown in FIG. 6. In this example embodiment, the anchoring member 368 is in the form of an inflatable balloon; however, it should be understood that other types of anchoring devices (e.g., expandable devices) can alternatively be used. For example, various anchoring devices are described in U.S. Patent Publication No. 2005/0234425 (the '425 publication), the disclosure of which is hereby incorporated by reference in its entirety.

The anchoring cannula 364 includes a first lumen 372 (see FIG. 7) that can be used to communicate an inflation medium to the anchoring member 368. The anchoring cannula 364 also includes a second lumen 370 that can be used to provide access to the interior of the intervertebral disc. For example, a delivery needle 362 can be inserted through the second lumen 370 of the cannula 364 and into the intervertebral disc D. The delivery needle 362 can be used to inject an enzyme (e.g., subtilisin) into the interior of the intervertebral disc D in a manner as described previously.

The anchoring member 368 allows the cannula 364 to maintain its position relative to the intervertebral disc D during a time period in which the enzyme (e.g., subtilisin) is dissolving portions of the nucleus N of the intervertebral disc D. As described previously, the time period for dissolution of the enzyme can vary depending on the particular situation. For example, after the enzyme has been injected into the interior of the disc D, the delivery needle 362 can be removed from the cannula 364. Maintaining the position of the cannula 364 enables multiple procedures to be performed within the intervertebral disc without having to reenter the disc for each procedure. For example, after a desired time period of dissolution, another device, such as a suction device (not shown) as described previously, can optionally be inserted through the lumen 370 and used to remove dissolved nucleus pulposus and the enzyme from the disc D

The methods of injecting an enzyme within an intervertebral disc and removing dissolved portions of nucleus pulposus (along with the enzyme) can be performed in conjunction with other methods of removing nucleus pulposus from an intervertebral disc. For example, the various methods and procedures described above can first be performed to dissolve a portion of nucleus pulposus. Another device configured to remove nucleus pulposus can subsequently be inserted into the interior of the disc and used to disrupt additional portions of the nucleus pulposus. Such devices can include, for example, one or more cutting portions configured to disrupt or tear nucleus pulposus tissue within the interior of the disc. Examples of devices configured to disrupt tissue are described in U.S. patent application Ser. No. 11/450,874 (the '874 application) and U.S. patent application Ser. No. 11/450,878 (the '878 application), the disclosures of which are hereby incorporated by reference in their entirety.

The combined dissolved and disrupted portions of nucleus pulposus tissue can then be removed using various different methods. For example, in some embodiments, a suction device (e.g., as described herein) can be used to suction the dissolved and disrupted portions of nucleus pulposus from the interior of the disc. In other embodiments, a different type of removal device can be used, such as a mechanically operated device that can grab or capture the disrupted and dissolved tissue and remove it from the interior of the disc. In some embodiments, the device used to disrupt the additional portions of nucleus pulposus can also be configured to remove the disrupted and dissolved tissue. For example, the device used to disrupt the tissue can be configured to provide suction through a lumen of the device. In another example, the device used to disrupt the additional portions of nucleus pulposus can be configured to remove the disrupted and dissolved tissue using means other than suction force (e.g., mechanically operated to capture the tissue). Examples of devices configured to both disrupt and remove tissue are described in the '874 application and the '878 application incorporated by reference above.

In some embodiments, a method and devices used to provide access to an interior of an intervertebral disc D includes the use of a guidewire. Such methods are described in the '425 publication incorporated by reference above. In one example, an introducer device and a pointed obturator are introduced together through the skin of a patient's back and distal ends of the devices are positioned near the intervertebral disc. The introducer device and the obturator can have any suitable dimensions, but in one embodiment introducer device is about 18-22 gauge and the obturator is about 20-25 gauge. The obturator can then be removed, leaving introducer device in place.

A needle can then be passed through introducer device and through the annulus fibrosis of the intervertebral disc to position its distal tip in the nucleus pulposus. The position of the introducer device and/or the needle can be confirmed using an imaging device, such as for example, an x-ray or fluoroscopy. In some embodiments, when needle is positioned in the nucleus pulposus, a contrast dye can be injected through needle, and the appearance of the contrast dye in the disc, as well as the patient's response to the injection, can be monitored. Such a procedure is generally known as a discography procedure. In alternative embodiments, discography may be performed at a later time or no discography may be performed.

After placing the needle within the intervertebral disc, a guidewire can be passed through a lumen of the needle and into the disc. The needle can then be removed, and a catheter device can be passed over the guidewire and through the introducer device. The catheter device can include two or more tubes or lumens, such as a guidewire tube and an injection or inflation tube, which can be configured to separate at a proximal end to attach, for example, to multiple adapters. Once the catheter device is in place, the introducer device can be removed and adapters can optionally be coupled to the proximal ends of the catheter tubes. The adapters can be used, for example, to facilitate guidewire passage, inflation of an expandable member, and/or injection of one or more substances into the intervertebral disc

One or more anchoring members such as anchoring member 368 described above, can be disposed on the catheter and deployed within the intervertebral disc to maintain a distal end portion of the catheter within the intervertebral disc. As described above, the anchoring member can be a variety of different configurations, such as an expandable balloon, or a mechanically actuated expandable device. Once the anchoring member is deployed, the guidewire can be removed. In some embodiments, an additional expandable member may be deployed outside the patient's body. With the anchoring member and the distal portion of catheter in place in the intervertebral disc, an enzyme can be introduced into the intervertebral disc through catheter in any of the methods described herein.

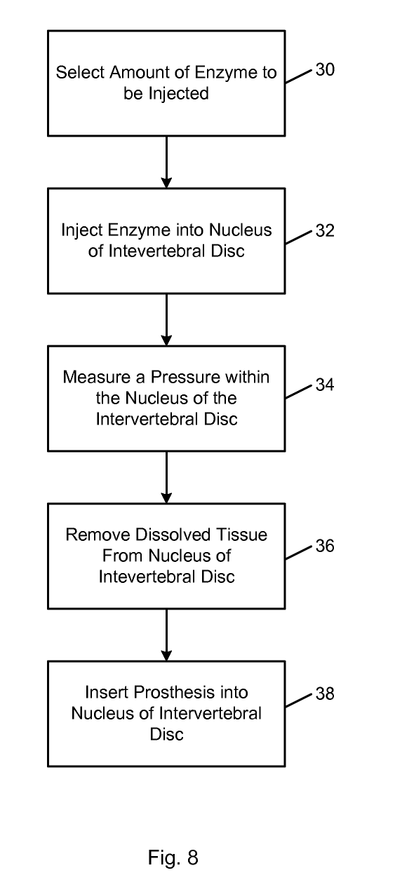

FIG. 8 is a flowchart illustrating a method for dissolving at least a portion of an intervertebral disc nucleus, according to an embodiment of the invention. A method includes at 30, selecting an amount of enzyme, such as a serine protease having an antigenicity less than an antigenicity of chymopapain to be injected into an interior portion (nucleus) of an intervertebral disc based on a desired amount of nucleus pulposus tissue to be removed. The selection process can also include selecting one or more buffers or carrier solutions to combine with the selected enzyme as described herein. The projected amount of nucleus pulposus tissue to be removed may be determined through various imaging techniques such as a magnetic resonance imaging (MRI) device. For example, three-dimensional (3D) imaging can be used to determine the condition and/or the volume of the nucleus. In another variation, a biopsy of the intervertebral disc can be taken prior to the procedure to determine the amount and/or concentration of the serine protease to be utilized in the procedure. In one implementation, a serine protease having an ability to sensitize less than an ability to sensitize of chymopapain is utilized in the procedure. The serine protease can be, for example, subtilisin.

At 32, the enzyme (e.g. serine protease) can be injected into the nucleus of the intervertebral disc. For example, any of the various methods and devices described herein can be used to gain access to an interior of an intervertebral disc, and then inject the enzyme within the nucleus of the intervertebral disc. A pressure within the nucleus of the intervertebral disc can optionally be measured at 34. The enzyme and dissolved portions of nucleus tissue can be removed at 36. For example, in some embodiments, after the pressure reaches a specified pressure threshold value, it may be desirable to remove nucleus pulposus tissue that has been dissolved by the enzyme. Alternatively, the tissue can be removed after a specified time period has lapsed after injecting the enzyme into the nucleus. The dissolved tissue can be removed, for example, using a device that suctions the dissolved tissue, or with other known removal devices as described herein. At 38, a prosthesis can optionally be disposed within a cavity formed in the nucleus by the removal of the dissolved tissue. Alternatively, hydrogel, biocompatible polymer, or other suitable material can optionally be injected into the cavity. In some embodiments, other suitable materials can be living cells, transplanted tissue, and/or engineered tissue. Other materials that can be injected (for example, in combination with a prosthesis, hydrogel, polymer, cells, and/or tissue) include bioactive agents, such as growth factors, anti-inflammatory drugs, analgesic (pain-killing) drugs, and genetic materials (i.e., molecules to genetically modify resident cells or added cells).

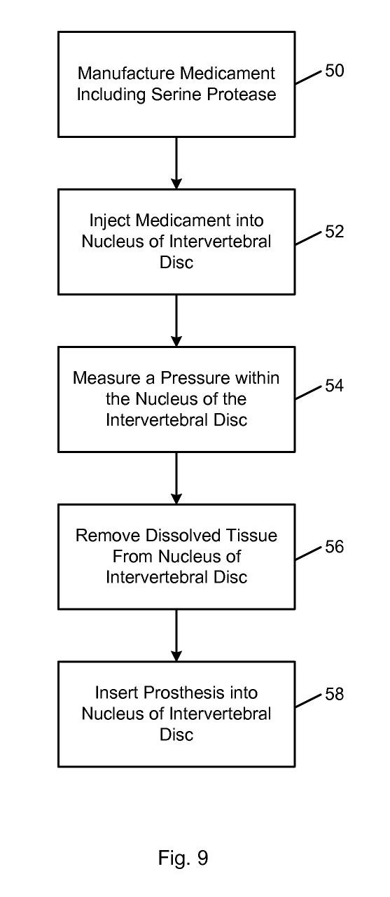

In another embodiment illustrated in FIG. 9, a use of a serine protease having an antigenicity less than an antigenicity of chymopapain for the manufacture of a medicament for treatment of an intervertebral disc is described. In one variation, the serine protease has an ability to sensitize less than an ability to sensitize of chymopapain. The serine protease can be, for example, subtilisin. A medicament including such a serine protease can be manufactured at 50. The medicament can be administered by delivering the medicament into a nucleus of an intervertebral disc at 52 using any of the various methods and devices described herein. For example, the medicament can be delivered by a minimally-invasive or percutaneous procedure, such as with a syringe. The medicament is configured to dissolve at least a portion of nucleus pulposus tissue within the intervertebral disc. A pressure within the nucleus of the intervertebral disc can optionally be measured at 54. At 56 nucleus pulposus tissue that has been dissolved by the medicament and remaining medicament can be removed from the nucleus of the intervertebral disc. For example, as stated above, when the pressure within the intervertebral disc is at a specified threshold value, it may be desirable to remove the dissolved tissue. Alternatively, the dissolved tissue and remaining medicament can be removed after a specified time period has lapsed after injecting the medicament into the nucleus. The dissolved tissue can be removed using any of the methods and devices described herein or other known removal devices not specifically described. For example, tissue can be removed using a device configured to suction the dissolved tissue. At 58, a disc prosthesis can optionally be disposed within a cavity formed in the nucleus by the removal of the dissolved tissue. Alternatively, hydrogel, or other suitable material can optionally be injected into the cavity. As stated above, in some embodiments, other suitable materials can be living cells, transplanted tissue, and/or engineered tissue. Other materials that can be injected (for example, in combination with a prosthesis, hydrogel, polymer, cells, or tissue) include bioactive agents, such as growth factors, anti-inflammatory drugs, analgesic (pain-killing) drugs, and genetic materials (i.e., molecules to genetically modify resident cells or added cells).

The methods described herein can be performed using known medical devices configured for such procedures. For example, a variety of different delivery device or syringe configurations can be used for injecting the enzyme (e.g., serine protease or related medicament) into the intervertebral disc. In addition, a variety of different known devices can be used that provide suction for the removal of dissolved tissue and/or the serine protease and/or other tissue from within the interior of the intervertebral disc. A variety of different types of pressure detection devices can also be used. Various configurations of a cannula or catheter device can also be used. For example, a cannula or catheter can have one or more lumens. The lumens of a cannula or catheter can also have a variety of different shapes (e.g., round, elliptical, oval, square, rectangular, triangular, etc.) and sizes.

While various embodiments of the invention have been described above, it should be understood that they have been presented by way of example only, and not limitation. It will be understood that various changes in form and details may be made. Where method and steps described above indicate certain events occurring in certain order, those of ordinary skill in the art having the benefit of this disclosure would recognize that the ordering of certain steps may be modified and that such modifications are in accordance with the variations of the invention. Additionally, certain of the steps may be performed concurrently in a parallel process when possible, as well as performed sequentially as described above.

For example, in some embodiments, the enzyme (e.g., a serine protease having a antigenicity less than an antigenicity of chymopapain and/or an ability to sensitize that is less than an ability to sensitize of chymopapain), or related medicament can remain in the patients body indefinitely. In other embodiments, the enzyme (e.g., serine protease, or medicament) is removed from the patient's body. In addition, the amount of serine protease or the amount of the serine protease included in a medicament can vary depending on the particular medical need and/or use.

The enzyme, such as, a serine protease having an antigenicity less than an antigenicity of chymopapain, and/or an ability to sensitize less than an ability to sensitize of chymopapain, can be used as a single procedure to treat an intervertebral disc, without any additional medical procedures. Alternatively, the injection of an enzyme as described herein can be used in combination with other procedures. For example, the procedures described herein can be used prior to inserting a disc prosthesis. A procedure including the use of such a serine protease can be performed immediately prior to a subsequent procedure, or at a time much earlier than a subsequent procedure.

A procedure to inject an enzyme as described herein can include various combinations or sub-combinations of the methods and devices described herein. For example, although one method of gaining access to an interior of an intervertebral disc may have been described with respect to a particular embodiment, such methods can also be used with other embodiments. Similarly, various combinations of the different medical devices (e.g., syringes, needles, delivery devices, suction devices, catheters, cannulas, guidewires, etc.) used for various portions of the procedures can be used. For example, a procedure including the use of an endoscope can be included in any of the procedures described herein. In another example, the use of radiopaque agents or markers can be included in any of the procedures described herein.

In addition, although an embodiment may have been described only the injection of an enzyme, it should be understood, that in any of the described embodiments, one or more buffers or carriers can be injected into the interior of an intervertebral disc as described herein. For example, a buffer can be combined with an enzyme prior to, or after, the enzyme is injected into an intervertebral disc. A buffer can also be injected simultaneously with the enzyme such that they are combined as they are being introduced into the intervertebral disc. Thus a buffer can be added before, during or after the injection of the enzyme into the intervertebral disc.

What is claimed is:

1. A method, comprising:
injecting a serine protease having an antigenicity less than an antigenicity of chymopapain into an interior portion of an intervertebral disc, the serine protease configured to dissolve tissue within the interior portion of the intervertebral disc; and
removing tissue that is dissolved by the serine protease from the interior portion of the intervertebral disc after the injecting.

2. The method of claim 1, wherein the serine protease has an ability to sensitize that is less than an ability to sensitize of chymopapain.

3. The method of claim 1, wherein the removing includes percutaneously suctioning the tissue dissolved by the serine protease from the interior portion of the intervertebral disc.

4. The method of claim 1, wherein the removing occurs after a time period no less than 5 minutes.

5. The method of claim 1, wherein the injecting includes percutaneously injecting the serine protease with a syringe having a needle with a size within a range of 6 gauge and 20 gauge.

6. The method of claim 1, further comprising:
measuring a pressure within the interior portion of the intervertebral disc prior to the removing, the removing being performed when the measured pressure crosses a pressure threshold.

7. The method of claim 1, further comprising:
determining a condition within the intervertebral disc prior to the removing tissue.

8. The method of claim 1, further comprising:
determining whether a nucleus within the interior portion of the intervertebral disc has been at least partially dissolved prior to the removing tissue.

9. The method of claim 1, further comprising:
inserting a prosthesis into the interior portion of the intervertebral disc after the removing tissue.

10. The method of claim 1, further comprising:
injecting a material into the interior portion of the intervertebral disc after the removing tissue.

11. The method of claim 1, further comprising:
prior to the injecting, selecting an amount of the serine protease based on a predetermined amount of tissue to be dissolved,
the injecting including injecting the amount of the serine protease.

12. The method of claim 1, wherein the serine protease is formulated with a contrast medium such that a location of the serine protease after the injecting can be determined with an imaging device.

13. The method of claim 1, wherein the serine protease is subtilisin.

14. A kit, comprising:
a medicament including a serine protease having an ability to sensitize less than an ability to sensitize of chymopapain and configured to dissolve tissue within a nucleus of an intervertebral disc; and
a delivery device configured to be percutaneously inserted into the nucleus of an intervertebral disc and inject the medicament into the nucleus of the intervertebral disc.

15. The kit of claim 14, wherein the delivery device includes a syringe.

16. The kit of claim 14, wherein the delivery device includes a needle having a size within a range of 6 gauge and 20 gauge.

17. The kit of claim 14, further comprising:
a pressure sensor configured to measure a pressure within a nucleus of the intervertebral disc.

18. The kit of claim 14, wherein the medicament including the serine protease includes subtilisin.

19. The kit of claim 14, further comprising:
a vacuum configured to suction collectively from the nucleus of the intervertebral disc tissue that has been dissolved by a first portion of the medicament and a second portion of medicament that has been injected into the nucleus of the intervertebral disc.

20. The kit of claim 14, wherein the serine protease has an antigenicity less than an antigenicity of chymopapain.

21. A structure, comprising:
an annulus of an intervertebral disc; and
a nucleus of an intervertebral disc at least partially bounded by the annulus of the intervertebral disc, the nucleus including nucleus pulposus dissolved through surgical intervention with a delivery of a serine protease having an antigenicity less than an antigenicity of chymopapain into the nucleus of the intervertebral disc.

22. The structure of claim 21, wherein the nucleus includes a first portion with the nucleus pulposus dissolved through surgical intervention and a second portion with undissolved nucleus pulposus.

23. The structure of claim 21, wherein the structure defines a volume having the dissolved nucleus pulposus and bounded by the annulus.

24. The structure of claim 21, wherein the structure defines a volume having the dissolved nucleus pulposus and bounded by the annulus, the structure further comprising:
a portion of the serine protease remaining after the nucleus pulposus has been dissolved and disposed within the volume.

25. The structure of claim 21, wherein the nucleus includes a first portion with the nucleus pulposus dissolved through surgical intervention and a second portion with undissolved nucleus pulposus, the structure defines a volume having the first portion of the nucleus pulposus and bounded by at least one of the second portion of the nucleus pulposus or the annulus.

26. The structure of claim 21, wherein the serine protease is subtilisin.

27. Use of a serine protease having an antigenicity less than an antigenicity of chymopapain for manufacture of a medicament for treatment of an intervertebral disc, the medicament being administered by:
delivering the medicament into a nucleus of an intervertebral disc, the delivery being minimally invasive, the medicament configured to dissolve at least a portion of pulposus within the nucleus of the intervertebral disc; and
removing collectively the dissolved pulposus and a remaining portion of the medicament from the nucleus of the intervertebral disc after the injecting.

28. The use of claim 27, wherein the removing occurs after a time period no less than 5 minutes.

29. The use of claim 27, wherein the delivering includes delivering the medicament with a syringe having a needle with a size within a range of 6 gauge and 20 gauge.

30. The use of claim 27, further comprising:
measuring a pressure within the nucleus of the intervertebral disc prior to the removing, the removing being performed after the pressure crosses a pressure threshold.

31. The use of claim 27, further comprising:
inserting a prosthesis into the nucleus of the intervertebral disc after the removing.

32. The use of claim 27, wherein the serine protease has an ability to sensitize less than an ability to sensitize of chymopapain.

33. The use of claim 27, wherein the serine protease is subtilisin.
